(19) United States
(12) Patent Application Publication
(10) Pub. No.: US 2010/0291225 A1
(43) Pub. Date: Nov. 18, 2010

(54) Stabilized Sustained Release Composition of Bupropion Hydrochloride and Process For Preparing the Same

(75) Inventors: Anuj Kumar Fanda, Uttar Pradesh (IN); Maulik Kiritkumar Panchal, Gujarat (IN); Gour Mukherji, Haryana (IN)

Correspondence Address: COHEN, PONTANI, LIEBERMAN & PAVANE LLP, 551 FIFTH AVENUE, SUITE 1210, NEW YORK, NY, 10176, US

(73) Assignee: Jubilant Organosys Ltd., Noida, Uttar Pradesh (IN)

(21) Appl. No.: 12/812,693

(22) PCT Filed: Nov. 9, 2008
(86) PCT No.: PCT/IN08/00777

§ 371 Date: Jul. 13, 2010

(30) Foreign Application Priority Data
Jan. 14, 2008 (IN) 117/DEL/2008

(51) Int. Cl. A61K 9/52; A61K 9/00; A61K 31/135; A61P 25/34; A61P 25/24; A61K 9/14

(52) U.S. Cl. 424/498; 424/400; 514/649; 424/489

(57) ABSTRACT

Disclosed herein is a stabilized sustained release pharmaceutical composition of bupropion hydrochloride and process for preparing the same, wherein said pharmaceutical composition comprising therapeutically effective amount of uncoated fine bupropion hydrochloride and pharmaceutically acceptable adjuvants, and wherein the composition is free of an acidic stabilizer and contains less than about 0.3% by weight of m-chlorobenzoic acid.

### FIELD OF THE INVENTION

The present invention relates to a field of sustained release pharmaceutical compositions in general, and in particular to a stabilized sustained release pharmaceutical composition of bupropion hydrochloride and a process for preparing the same.

### BACKGROUND OF THE INVENTION

Bupropion hydrochloride is an antidepressant of the aminoketone and a non-nicotine aid to smoking cessation; is chemically unrelated to tricyclic, tetracyclic, selective serotonin reuptake inhibitor, or other known antidepressant classes. Bupropion described in U.S. Pat. Nos. 3,819,706 and 3,885,046 is currently available as the hydrochloride salt and chemically it is (±)-1-(3-chlorophenyl)-2-[(1,1-dimethylethyl)-amino]-1-propranone hydrochloride. Bupropion is marketed as a sustained release tablet formulation under the brand name Wellbutrin®SR, by Glaxosmithkline.

Bupropion hydrochloride is a degradation prone product when formulated with conventional pharmaceutical excipients into solid dosage forms. Though exact mechanism of degradation has not been fully elucidated, literature and patent information seem to indicate that hydrolysis and oxidation are the possible mechanisms of degradation. The main degradation product is m-chlorobenzoic acid.

Well-known methods for stabilization generally involved use of acid stabilizers to create acidic environment in the vicinity of bupropion particles.

U.S. Pat. Nos. 5,358,970; 5,541,231; 5,731,000 and 5,763,493 described a stabilized bupropion hydrochloride formulation having a stabilizer selected from group consisting of L-cysteine hydrochloride, glycine hydrochloride, malic acid, sodium metabisulfite, citric acid, tartaric acid, L-cystine dihydrochloride, ascorbic acid, and isoascorbic (erythorbic) acid.

Stabilization by acidification of the environment in which degradation occurs in pharmaceutical compositions containing bupropion is disclosed in U.S. Pat. No. 5,968,553. In this patent, the stabilizer is an inorganic acid having an aqueous solution pH of about 0.5 to 4.0 at a concentration of about 0.31% w/w. The inorganic acids are selected from the group consisting of hydrochloric acid, phosphoric acid, nitric acid and sulfuric acid.

U.S. Pat. No. 7,241,805 assigned to Biovail discloses a modified release bupropion hydrobromide tablet for oral administration with enhanced stability. Said tablet composition either involves use of stabilizer in the core or enteric coating over the core, which has stabilizing effect.

Sustained release tablet forms of bupropion are described in U.S. Pat. No. 5,427,798, comprising a sustained release tablet where sustained release of drug is achieved by combining bupropion particles, microcrystalline cellulose with hydrogel-forming high molecular weight, high viscosity grades of hydroxypropyl methylcellulose. Stabilization of this formulation is taught by addition of cysteine hydrochloride or glycine hydrochloride.

Another method for stabilization includes formation of barrier layer surrounding bupropion or excipients, with water soluble or insoluble polymers, thereby physically separating excipients from bupropion. Further methods involve the use of large particle size bupropion particles and exclusion of wet granulation techniques during manufacturing.

U.S. Pat. No. 6,306,436 assigned to Teva discloses stabilized bupropion hydrochloride pharmaceutical compositions that are free of added acid and provide for sustained release of bupropion hydrochloride. Stabilization is achieved by coating the particles of bupropion hydrochloride, or by using large particle size bupropion crystals (75-900 μm). Although said patent avoids the potential disadvantages of using an acid, the disclosed invention requires drug particle coating, which is essentially an expensive and time-consuming process.

U.S. Pat. No. 6,893,660 assigned to Andrx discloses a pharmaceutical composition in solid form comprising pharmaceutically active ingredients combined with excipients having a negative impact on stability of the active ingredients. The said patent encompasses seal coating of the excipients in order to separate active ingredients from the negative effect of the excipients thus unlike the prior art, said patent favorably influences stability by physically sealing the excipients rather than chemically adjusting pH. The invention requires a cumbersome and relatively expensive process of coating of the excipients.

U.S. Pat. No. 6,238,697 assigned to Pharmalogix discloses a process of manufacturing extended release bupropion hydrochloride tablets using direct compression without employing a slugging or wet granulation technique. Bupropion particles having particle size in the range of 130 to 450 μm is the preferred embodiment.

US Application No. 2003/0044462 assigned to Kali labs discloses a solid composition comprising a bupropion hydrochloride and carbovinyl polymer. Said polymer acts as a stabilizer as well as sustained release polymer. It is also disclosed that the main degradation product of bupropion hydrochloride is m-chlorobenzoic acid.

US Application No. 2006/0165729 assigned to Ranbaxy labs discloses a tablet composition comprising a bupropion hydrochloride, which is free of stabilizer. Said tablet composition is prepared by dry granulation method. It is also disclosed that the wet granulation is not advisable for bupropion hydrochloride, because of its hygroscopic nature and its high susceptibility to decomposition.

US Application No. 2006/0204571 teaches formulation of bupropion hydrochloride using talc and potassium chloride. Such a formulation is capable of preventing degradation of bupropion hydrochloride.

International Publication No. WO2007/060540, assigned to Aurobindo Pharma, discloses stable bupropion hydrochloride composition with low amount of hydroxypropyl methylcellulose phthalate as a stabilizer.

Although various prior arts teach stabilization of bupropion formulations either by incorporating acidic additives or by avoiding direct contact between bupropion and excipients with the help of coating, all these procedures are less desirable because it represents additional ingredients or additional steps in the processing of the formulation. The need still exists in the art, for a stable sustained release bupropion formulation that controls the level of degradation products without recourse to an acid stabilizer or coating of drug or excipients. Such sustained delivery dosage formulations have a practical application, and represent a valuable contribution to the pharmaceutical arts. The present invention provides such compositions, and offers efficient and cost effective methods of preparation.

Pharmaceutical compositions suitable for oral administration to mammals and containing bupropion hydrochloride should have constant chemical and physical properties in accordance with enacting health registration requirements of U.S and international health registration authorities, e.g., the FDA's Good Manufacturing Practices (“GMP”) requirements and international conference on Harmonization (ICH) guidelines.

It has been observed that it is relatively easier to preserve the assay value of the active ingredient in the formulation within the specified range as prescribed by regulatory bodies such as USFDA, but it is a really challenging task to control the level of degradation product, particularly m-chlorobenzoic acid, in the final formulation.

Accordingly, development of an effective pharmaceutical composition of bupropion hydrochloride with low levels of m-chlorobenzoic acid is highly desired which can replace the above mentioned stabilization method, which requires the use of acid stabilizers, particle coating or use of larger particle size bupropion.

The present invention provides a stabilized pharmaceutical composition of bupropion hydrochloride, possessing marked improvement in stability over the shelf life, employing selective bupropion particle size, processing and packaging conditions.

### SUMMARY OF THE INVENTION

In accordance with the principal aspect of the present invention, there is provided a stable oral sustained release pharmaceutical composition comprising bupropion hydrochloride that exhibits sustained release of the drug.

In accordance with another aspect of the invention, there is provided a process for preparing said stable oral sustained release pharmaceutical composition comprising bupropion hydrochloride that exhibits sustained release of the drug.

In accordance with one other aspect of the present invention, there is provided a stable oral sustained release pharmaceutical composition comprising therapeutically effective amount of uncoated fine bupropion hydrochloride and pharmaceutically acceptable adjuvants, wherein the composition is free of an acidic stabilizer and contains less than about 0.3% by weight of m-chlorobenzoic acid when stored at 40° C.±2° C. and 75%±5% RH for 3 months in specialized packs.

In accordance with one other aspect of the present invention, there is provided a stable oral sustained release pharmaceutical composition comprising therapeutically effective amount of uncoated fine bupropion hydrochloride and pharmaceutically acceptable adjuvants, wherein said composition contains less than about 0.3%, preferably less than about 0.2% of degradation product such as m-chlorobenzoic acid, when stored at 40° C.±2° C. and 75%±5% RH for three months in specialized packs.

In accordance with one other aspect of the present invention, there is provided a stable oral sustained release pharmaceutical composition comprising therapeutically effective amount of uncoated fine bupropion hydrochloride and pharmaceutically acceptable adjuvants, wherein the composition is free of an acidic stabilizer and contains less than about 0.3% by weight of m-chlorobenzoic acid when stored at 40° C.±2° C. and 75%±5% RH for three months in specialized packs and wherein the said composition is prepared under controlled processing conditions.

In accordance with yet another aspect of the present invention, there is provided a stable oral sustained release pharmaceutical composition comprising a compressed core having therapeutically effective amount of fine bupropion hydrochloride and one or more pharmaceutically acceptable adjuvants, wherein said core is necessarily free of stabilizer and contains uncoated fine particles of the bupropion hydrochloride, having particle size approximately in the range of about 1 to 100 μm.

In accordance with yet another aspect of the present invention, there is provided a stable oral sustained release pharmaceutical composition of bupropion hydrochloride for oral administration, wherein said composition contains at least about 80% of undegraded bupropion hydrochloride after storage for three months at 40° C.±2° C. (temperature), and 75%±5% RH (relative humidity) for three months in specialized packs.

In accordance with yet another aspect of the present invention, there is provided a stable sustained release pharmaceutical composition of bupropion hydrochloride for oral administration wherein the composition is manufactured in the controlled processing conditions.

In accordance with yet another aspect of the present invention, there is provided a stable oral sustained release pharmaceutical composition of bupropion hydrochloride for oral administration, wherein the composition is prepared under controlled processing conditions comprising the steps of: (a) preparing a core, (b) optionally, forming a seal coating layer on the core and (c) optionally, forming a film coating on the seal coated core, and (d) packing final product in specialized packs.

In accordance with yet another aspect of the present invention, there is provided a stable sustained release pharmaceutical composition of bupropion hydrochloride for oral administration, wherein the composition is prepared under controlled processing conditions comprising the steps of: (a) preparing the core comprising (i) sifting bupropion hydrochloride and lipophilic release retardant material(s); (ii) sifting channeling agent(s) and swelling enhancer(s) and optionally other pharmaceutical adjuvant (s), (iii) preparing a binder solution in either aqueous or non-aqueous solution, (iv) mixing the blend of step (i) geometrically with blend of step (ii), (v) granulating the resultant blend employing aqueous or non-aqueous solvent, (vi) drying the resultant granulates, (vii) lubricating the dried granulates with lubricant, (viii) compressing the lubricated granulates into a compressed core, (b) optionally forming the seal coating layer on the core, (c) optionally forming the non-functional film coating on the seal coated core and (d) packing the resultant product in specialized packs.

In accordance with yet another aspect of the present invention, there is provided a stable sustained release pharmaceutical composition of bupropion hydrochloride for oral administration, wherein the composition is formulated in various oral delivery devices, preferably tablet, capsule, granules, beads, or sachet.

The details of one or more embodiments of the inventions are set forth in the description below. Others features, objects and advantages of the inventions will be apparent from the description and claims.

### DESCRIPTION OF THE INVENTION

The present invention describes a stabilized sustained release pharmaceutical composition of bupropion hydrochloride having a desired drug release profile to provide the twice a day dosage composition and the process for preparing the same. The process for preparing such a composition is efficient, simple and easy to scale up.

“Sustained-release pharmaceutical compositions” or dosage forms which exhibit a “sustained-release” of the bupropion salt as used herein is defined to mean pharmaceutical composition administered twice-daily that provide a release of the bupropion salt sufficient to provide a therapeutic dose following its administration, and then a gradual release over an extended period of time such that the sustained-release dosage form provides therapeutic benefit over a 12 or 14-hour period.

“Non-functional coatings” are coatings that do not affect drug release, but which affect other properties, such as the enhancement of the chemical, biological or physical stability characteristics, or the enhancement of the aesthetic appeal of the pharmaceutical composition.

The term “bupropion hydrochloride” is used to refer to the hydrochloride salt of the m-chloro-α-(t-butylamino) propiophenone.

“Controlled processing conditions” as described herein refers to environmental conditions during manufacturing of composition of the present invention, are strictly monitored and maintained at a relative humidity in the range of about 25%±5% and temperature in the range of about 25° C.±5° C.

“Specialized packaging or pack” as used herein refers to packaging or pack for storage, distribution and protection from external environmental conditions and mechanical stress of transportation and said pack may be made up of HDPE bottles, various types of blister or similar pharmaceutically acceptable packing material. Said pack is provided with additional aids like dessicants, molecular sieves, canisters, silica gel, nitrogen flushing or other suitable means to ensure stability of composition of the invention during its storage.

One of the surprising aspect of the invention is that bupropion hydrochloride is required to be in fine crystals with particle size approximately in the range of about 1 to 100 μm, preferably in the range of about 5 to 90 μm, more preferably in the range of about 10 to 75 μm, still more preferably in the range of about 20 to 65 μm. It is found according to the present invention that if the said critical particle size of bupropion is used, it is not necessary to add acid stabilizer to stabilize the sustained release pharmaceutical composition. In a preferred embodiment of the invention, bupropion hydrochloride crystals having a particle size in the range of approximately 1 to 100 μm are used to provide a stabilized composition. The tablets of the present invention comprising small particle size bupropion hydrochloride, have unexpected enhanced stability compared to the prior art bupropion hydrochloride tablets.

One embodiment of the present invention encompasses stabilized sustained release pharmaceutical composition of bupropion hydrochloride comprising uncoated fine particles of bupropion having particle size approximately in the range of about 1 to 100 μm wherein the main degradation product i.e. m-chlorobenzoic acid is preferably restricted in amounts of less than 0.3% when the composition is stored at 40° C.±2° C. (temperature), and 75%±5% RH (relative humidity) for three months.

Another embodiment of the present invention encompasses stabilized sustained release pharmaceutical composition of bupropion hydrochloride comprising uncoated fine particles of bupropion having particle size approximately in the range of about 1 to 100 μm wherein said composition contains at least about 80%, preferably 85%, more preferably 95% of undegraded bupropion hydrochloride after storage at 40° C.±2° C. (temperature) and 75%±5% RH relative humidity for three months in specialized packs.

One aspect of the present invention relates to a stabilized sustained release pharmaceutical composition comprising a compressed core containing bupropion hydrochloride and one or more pharmaceutically acceptable adjuvants, optionally coated with seal and film coating, wherein said core is necessarily free of acid stabilizer and contains uncoated fine crystals of the bupropion hydrochloride having particle size approximately in the range of about 1 to 100 μm.

Still another embodiment of the present invention describes a stabilized sustained release pharmaceutical composition comprising a compressed core containing fine uncoated bupropion hydrochloride crystals, one or more pharmaceutically acceptable adjuvants and necessarily free of acid stabilizer, followed by seal coating, then with non-functional film coating wherein the main degradation product i.e. m-chlorobenzoic acid is preferably present in amounts less than about 0.3%, preferably 0.2%, when the composition is stored at 40° C.±2° C. (temperature), and 75%±5% RH (relative humidity) for three months in specialized packs.

Still another embodiment of the present invention relates to a process of preparing the stabilized pharmaceutical composition comprising a compressed core containing fine uncoated bupropion hydrochloride crystals, one or more pharmaceutically acceptable adjuvants and necessarily free of acid stabilizer, coated with seal coating, then with non-functional film coating wherein the main degradation product i.e. m-chlorobenzoic acid is preferably present in amounts less than about 0.3% and contains at least about 80% of undegraded bupropion hydrochloride when the composition is stored at 40° C.±2° C. (temperature), and 75%±5% RH (relative humidity) for three months in specialized packs, wherein the composition is prepared under controlled processing conditions.

Another embodiment of the present invention relates to an oral stable pharmaceutical composition comprising a therapeutically effective amount of bupropion hydrochloride in the form of uncoated fine crystals having particle size in the range of about 1 to 100 μm, one or more lipophilic release retardants, one or more pharmaceutically acceptable adjuvants, wherein said composition contain less than about 0.3% of m-chlorobenzoic acid when stored for three months at 40° C.±2° C. and 75%±5% relative humidity in specialized packs, wherein said composition is necessarily free of acid stabilizer wherein composition is prepared preferably by wet granulation process under controlled processing conditions.

In an embodiment of the present invention, the pharmaceutical composition of bupropion hydrochloride may be in the form of granules, pellets, capsules, conventional tablets, sustained release tablets, controlled release tablets or extended release tablets. Preferably, the pharmaceutical composition of bupropion hydrochloride in the form of sustained or controlled release tablets.

One embodiment of the present invention encompasses a stabilized oral sustained release pharmaceutical composition comprising therapeutically effective amount of bupropion hydrochloride, suitable lipophilic release retardants, one or more pharmaceutically acceptable adjuvants like weight adjusting agents, channeling agents, binders, swelling enhancers, lubricants, glidants and optionally other pharmaceutically acceptable adjuvants, wherein said composition is necessarily free of acid stabilizers.

The composition of the present invention usually may contain 25 mg to 500 mg of bupropion hydrochloride. However, the exact dosage regimen will depend on a number of factors, including age, the general condition of the patient, the particular condition or disorder being treated, the severity of the patient's condition and the like.

The “adjuvants” used in accordance with the present invention include pharmaceutically acceptable compounds which are intended to enhance the handling and/or manufacturing of the pharmaceutical composition into an acceptably uniform, flowable and compressible admixture which can be readily converted into the final dosage form such as capsules, tablets and the like. By “pharmaceutically acceptable” is meant a material which is not biologically or otherwise undesirable, i.e., the material can be administered to an individual along with the active ingredient without causing any undesirable biological effects or interacting in a deleterious manner with any other components of the composition.

Those skilled in the art will appreciate that adjuvants may vary depending on the strength of particular adjuvants used and the level and the amount approved by regulatory authorities for use in pharmaceutical products.

Unlimited examples of lipophilic release retardants in accordance with the present invention include, but not limited to, hydrogenated oils such as, hydrogenated vegetable oil, cottonseed oil, castor oil, canola oil, palm oil, palm kernel oil and soybean oil, cetostearyl alcohol, cetyl alcohol, glyceryl behenate derivatives (such as Compritol® ATO888, Compritol® HD ATO5), glyceryl mono oleate, glyceryl mono stearates, glyceryl palmito stearates (such as Precirol® ATO5, lecithin, mono-di- and triglycerides with polyethylene glycol (PEG) esters of fatty acid (such as Gelucire® 54/02, 50/13, 43/01), medium chain triglycerides, carnauba wax, microcrystalline wax, beeswax, any combination thereof and the like. Other forms of sustained release agents are also contemplated. In particular, hydrogenated castor oil (commercially available under the brand name Cutina®HR from Cognis, North America) is found to be useful.

According to the present invention, the effective amount of lipophilic release retardant required to achieve sustained release of bupropion hydrochloride may vary between 25% to 55%, but preferably 30% and 50% and most preferably between 35% and 45% of the uncoated tablet weight. In general, any amount that will effectively demonstrate a sustained release profile of the bupropion hydrochloride can be used.

Unless otherwise stated all concentrations mentioned herein are based on total weight of the composition.

The ratio of bupropion hydrochloride to lipophilic release retardant material ranges from about 0.5 to 1.5. The concentration of lipophilic material used is reasonably equal, allowing formation of very hard tablets, which can withstand various rigors. It is believed, without wishing to be bound by any theory that, the use of lipophilic release retardant material having melting point higher than human body temperature contributes to the stability of the dosage form.

The artisan will appreciate that the desired in-vitro release rates described herein for the bupropion hydrochloride is achieved by controlling the release of drug from the core matrix. The diffusion or dissolution of the drug from the core can be altered by varying the ratio of bupropion hydrochloride to lipophilic material.

Weight adjusting agents also termed “fillers” are typically necessary to increase the bulk of the material to facilitate easy compression. Suitable weight adjusting agents include, for example, dicalcium phosphate dihydrate, calcium sulfate, lactose, cellulose, kaolin, mannitol, sodium chloride, dry starch, hydrolyzed starches, microcrystalline cellulose, powdered sugar, hydrogenated oils or any combination thereof and the like. The preferred diluent for the composition of the present invention is mannitol. For the present invention, preferably spray dried mannitol (commercially available under the brand name Pearlitol SD200 from Roquette, France) is useful to obtain tablet of desirable weight. The weight-adjusting agents may be present in the composition in an amount of from about 1% to about 70% by weight of the uncoated tablet, more particularly from about 2% to about 50%, preferably from about 3% to about 30%, still more preferably from about 5% to about 20% by weight of the uncoated tablet. Mannitol serves dual purposes, firstly as weight-adjusting agents and secondly as a channel-forming agent in the tablet core. A particular amount of manniitol can range from about 5% to about 20% by weight of the uncoated tablet. Likewise, other weight-adjusting agents are also considered to be used as channel forming agent in the composition in the range of about 1% to about 20% by weight of the uncoated composition.

Binders are used to impart cohesive qualities to tablet formulation, and thus ensure that a tablet remains intact after compression. Suitable binder materials include, but are not limited to, starch (including corn starch and pregelatinized starch), gelatin, highly dispersed silica, sugars (including sucrose, glucose, dextrose, lactose, mannitol and sorbitol), polyethylene glycol, polyvinylpyrrolidone, waxes, hydroxypropyl methylcellulose, hydroxypropyl cellulose and natural and synthetic gums (acacia, tragacanth, sodium alginate and veegum). Binder can be added to the formulation in different ways: (i) as a dry powder, which is mixed with other ingredients before wet agglomeration, (ii) as a solution, which is used as agglomeration liquid during wet agglomeration, and is referred to as a solution binder, and (iii) as a dry powder, which is mixed with other ingredients before compaction (referred to as dry binder). For the composition of the present invention, the preferred binder is polyvinylpyrrolidone. Polyvinylpyrrolidone is commercially available from BASF, Germany, under the brand name of Kollidon®K90. The binder may be present in an amount of 0.5% to about 40%, particularly about 0.75% to about 30%, and more particularly about 1% to about 20% by weight of the uncoated composition.

Swelling enhancers are members of a special category of excipients that swell rapidly to a large extent when placed in a liquid medium resulting in an increase in the size of the tablet. Examples of swelling enhancers include, but not limited to cross-linked polyvinyl pyrrolidone, low-substituted hydroxypropyl cellulose, microcrystalline cellulose, cross-linked sodium or calcium carboxymethyl cellulose, cellulose fiber, cross-linked polyacrylic acid, cross-linked Amberlite resin, alginates, colloidal magnesium-aluminum silicate, corn starch, rice starch, potato starch granules, pregelatinised starch, sodium carboxymethyl starch or any combination thereof and the like. Preferably in sustained release pharmaceutical compositions of present invention, the swelling enhancer is cross-linked polyvinyl pyrrolidone. Cross-linked polyvinylpyrrolidone is commercially available from ISP Tech, under the brand name of Polyplasdone®XL. The content of the swelling enhancer can be from about 0.5% to about 10% by weight of the uncoated composition. Swelling enhancer is present in an amount of about 1% to about 5%, preferably about 1.5% to about 3% by weight of the uncoated composition.

The artisan can choose an appropriate lubricant to prevent sticking and picking of the tablet material to the compression tooling. Examples of suitable lubricants include, for example, magnesium stearate, calcium stearate, stearic acid, glyceryl behenate, and polyethylene glycol, or any combination thereof and the like. A preferred lubricant is magnesium stearate. The lubricant can be present in an amount of from 0.1% to 10% by weight of the uncoated composition. Preferably, the lubricant is present in an amount of from 0.5 to 2.5% by weight of the uncoated composition.

Unlimited examples of glidants in accordance with the present invention include, for example, calcium silicate, magnesium silicate, colloidon silicon dioxide, magnesium stearate, any combination thereof and the like. A preferred glidant is colloidon silicon dioxide. The glidant can be present in an amount of from 0.1% to 10% by weight of the uncoated composition. Preferably, the lubricant is present in an amount of from 0.5 to 2.5% by weight of the uncoated composition.

The additional inert excipients are well known to the skilled artisan can be found in the relevant literature, for example in the Handbook of Pharmaceutical Excipients.

Suitable solvents used for preparing stable pharmaceutical composition of bupropion hydrochloride include water, ethanol, acetone, methylene chloride, methanol and isopropanol or a combination thereof.

In an embodiment of the present invention, manufacturing of the stabilized oral sustained release pharmaceutical composition of bupropion hydrochloride is carried out under the controlled processing conditions, wherein the relative humidity is maintained at about 25%±5% and temperature is maintained at about 25° C.±5° C. The composition of the present invention is preferably prepared by wet granulation techniques while other techniques of manufacturing such as fluid bed processing, dry granulation, direct compression or any other manufacturing technique known in the art, are also within the scope of the present invention.

The methods, processes, and compositions described herein may provide one or more of the following features. For example, the method is simple and produces compositions having good stability during storage and desired sustained release characteristics. The method can avoid the use of an acid stabilizer, coated bupropion hydrochloride particles, and larger sized bupropion hydrochloride crystals, thereby resulting in reduced costs. Higher particle size of bupropion particles can also lead to problems related to content uniformity.

In one general aspect of the present invention, the present invention provides process for preparing said sustained release pharmaceutical composition of bupropion hydrochloride. The steps for preparation include (a) preparing the compressed core, (b) optionally, forming the seal coating layer on the core and (c) optionally, forming the film coating on the seal coated core.

In one another aspect of the present invention, there is provided a process for preparation of sustained release pharmaceutical composition of bupropion hydrochloride. The steps of preparation include (i) dry mixing bupropion hydrochloride, lipophilic release retardant(s) and one or more pharmaceutical adjuvants, (ii) granulating the resultant blend with aqueous/non-aqueous solvent and drying the resultant granulates; (iii) lubricating the dried granules and converting them into appropriate composition; (iv) coating the said composition with seal coating layer; (v) followed by coating the seal coated composition with non-functional film coating layer.

In another aspect of the present invention, there is provided a process for preparation of such composition wherein the process comprises the steps of: (A) preparing the core comprising (i) sifting bupropion hydrochloride and lipophilic release retardant material(s), (ii) sifting channeling agent(s) and swelling enhancer(s) and optionally other pharmaceutical adjuvant (s), (iii) preparing a binder solution in either aqueous or non-aqueous solution, (iv) mixing the blend of step (i) geometrically with blend of step (ii), (v) granulating the resultant blend with aqueous or non-aqueous solvent, (vi) drying the resultant granulates, (vii) lubricating the dried granulates with lubricant, (viii) compressing the lubricated granulates into a compressed core, (B) forming the seal coating layer on the core and (C) forming the non-functional film coating on the seal coated core. (D) finally, packing final product in specialized packs.

Another aspect of the present invention includes a process for preparation of such composition, which comprises of the following steps: (i) sifting bupropion hydrochloride and hydrogenated oil(s) through a suitable sieve, (ii) sifting channeling agent (s) and swelling enhancer (s) and optionally other pharmaceutical adjuvant(s) through a suitable sieve, (iii) preparing a binder solution in either aqueous or non-aqueous solution; (iv) mixing the blend of step 1 geometrically with blend of step 2 in a suitable equipment, (v) granulating the resultant blend with aqueous/non-aqueous solvent, (vi) drying the resultant granulates in a suitable drier, (vii) lubricating the dried granules with previously sifted lubricant and mix together, (viii) formulating the mixture into a suitable core composition, (ix) coating the core composition with seal coating composition, (x) followed by coating the seal coated composition with non-functional film coating composition, (xi) finally packing final product in specialized packs wherein all the steps were carried out under the controlled processing conditions such as relative humidity is maintained at about 25%±5% and temperature is maintained at about 25° C.±5° C.

In another example of the present invention, a stabilized oral pharmaceutical composition can comprise a therapeutically effective amount of bupropion hydrochloride intimately blended with lipophilic release retardant(s) and other conventional pharmaceutically acceptable excipients, changed into oral dosage form, said oral dosage form may be coated with seal coating and further with non-functional film coating wherein m-chlorobenzoic acid is present in amounts less than 0.3% when the composition is stored at 40° C.±2° C. at 75%±5% relative humidity for three months in specialized package.

In another embodiment of the present invention, specialized packaging materials which protect the formulation from moisture and oxygen, contributes to the shelf life of the final product. For example, suitable packaging materials include light protected HDPE bottles, light protected glass bottles and the like. Packaging will include a desiccant pack. The container may be in the form of various types of blister packs to provide the desired protection, maintain product stability and integrity. It may be advisable to do packaging under nitrogen or argon to reduce the headspace oxygen. Preferably, the pharmaceutical compositions of bupropion hydrochloride of the present invention may be packed in HDPE container with molecular sieve sachets/canister and/or the container may be purged with nitrogen gas in order to provide oxygen free environment.

In another aspect of the present invention, an oral stable sustained release pharmaceutical composition comprising therapeutically effective amount of fine bupropion hydrochloride wherein the composition is free of an acidic stabilizer and wherein the said composition contains less than about 0.3% by weight of m-chlorobenzoic acid when stored at 40° C.±2° C. and 75%±5% RH for 3 months in specialized packs and wherein the said composition is manufactured under controlled processing conditions.

In another example of the present invention, a stabilized oral pharmaceutical composition can comprise bupropion hydrochloride, hydrogenated castor oil and other pharmaceutically acceptable excipients, preferably mannitol and polyvinyl pyrrolidone for controlling the rate of release of the active ingredient for twice a day dosage regimen. The compositions may be seal coated and followed by non-functional film coatings (with color for product identification) for enhancing aesthetic appeal.

The seal-coating layer of the present invention is water soluble in nature and is designed to disintegrate rapidly in an aqueous medium. The seal coating layer of the present invention comprises at least one water soluble polymer selected from the group comprising of polyethylene glycol, hydroxypropyl methylcellulose, polyvinyl pyrrolidone, hydroxy propylcellulose or any combination thereof and the like. The preferred polymer for seal coating layer of the present invention is low viscosity hydroxypropyl methylcellulose. Hydroxypropyl methylcellulose, having viscosity of three centipoise, is commercially available from ShinEtsu, Japan, under the brand name Pharmacoat 603. Preferably, the water-soluble polymers comprising seal coating layer are used in amounts of about 1% to 10% by weight of the coated composition.

The seal-coating layer may further comprise other additives like plasticizers, such as, propylene glycol, triacetin, polyethylene glycol, tributyl citrate and optionally anti-tacking agents, such as, calcium silicate, magnesium silicate, and colloidal silicon dioxide or talc. The preferred plasticizer for the seal-coating layer is polyethylene glycol 400. Polyethylene glycol is commercially available from BASF Pharma, Germany under the brand name Lutrol E 400. Preferably, plasticizer is used in amount of about 0.01% to 5% by weight of the coated composition.

Apart from plasticizers and anti-tacking agents as mentioned above, the seal coating layer may optionally contain buffers, colorants, opacifiers, surfactants or bases, which are known to those skilled in the art.

The dispersion for seal coating ingredients may be prepared in aqueous/non-aqueous solvent. The aqueous solvent comprises purified water or the like, and non-aqueous solvent comprises organic solvent like ethanol, methanol, dichloromethane, isoprapanol, ethyl acetate, acetone, dimethyl formamide, benzene ethyl lactate, glacial acetic acid etc. The seal-coating layer is applied on the core using any conventional coating pan, perforated coating pan, fluidized bed apparatus, or any other suitable coating apparatus known in the art to achieve desired weight gain. The skilled artisan knows, on the basis of his technical knowledge the optimization techniques of various processing parameters for the above-mentioned coating equipments.

Seal coated composition is preferably film coated with conventional coating materials such as a Opadry II or Opadry AMB, Hydroxypropyl methylcellulose and the like. According to the invention, the film coating acts as a moisture barrier. The film coating does not substantially affect the release rate of the bupropion hydrochloride from the composition, since the coating is instant dissolving type, which rapidly dissolves in the stomach. Film coatings carried out by deposition of one or more film-forming polymers resulting in coats that usually represent no more than about 2% to 5% by weight of the final coated composition. A film coating solution according to the invention preferably contains, in addition to the film-former, a plasticizer, a glidant, an opacifying agent or a coloring agent, and a solvent system is composed of aqueous, hydro-alcoholic or non-aqueous solvent mixture. Some film coating materials are readily available in the form of a premix with all the necessary ingredients required for achieving a smooth and uniform film.

In one embodiment of the present invention, the composition is coated until appropriate weight gain is achieved, for example about 1% to 10% w/w in the case of seal coating, and about 2% to 10% w/w in case of film coating. The compositions are dried or allowed to cure as needed at the end of each coating process. The operational parameters are maintained according to the manufacturing recommendations.

The preferred weight of the composition is 50 to 1000 mg, most preferably 100 to 500 mg.

The composition of the present invention is bioequivalent with the marketed composition of bupropion (Wellbutrin® SR as well as to Zyban®) in terms of parameters like dissolution, disintegration and bioavailability.

Mixing and granulation can be carried out in suitable apparatus like rapid mixer granulator (RMG) or fluid bed processor or any other suitable equipment designed for the same purpose. Drying of granules can be carried in fluid bed processor itself or other drying techniques can be employed such as tray drying, vacuum drying, flash drying etc. Mixing of dried granules with lubricants can be carried out in a suitable mixer like double cone or V-blender or Conta blender. The tableting of lubricated granules can be carried out in a suitable tableting machine equipped with suitable tooling. Coating of tablets can be carried out in a suitable coating apparatus like conventional coating pan or fluid bed processor.

The positive impact of stability of the sustained release pharmaceutical composition of bupropion hydrochloride described herein is evident from the results of tests performed to evaluate the level of m-chlorobenzoic acid present in 150 mg pharmaceutical composition through three months period under accelerated conditions (40° C.±2° C./75% RH±5% RH). The stability tests showed reduced values in terms of concentration of m-chlorobenzoic acid.

The pharmaceutical compositions disclosed herein contain at least about 80% of the drug after three months when stored at 40° C.±2° C. and 75%±5% relative humidity, preferably contains at least about 85% of the drug after three months when stored at 40° C.±2° C. and 75% relative humidity, more preferably contains at least about 90% of the drug after three months when stored at 40° C.±2° C. and 75%±5% relative humidity.

In yet another embodiment of the present invention, there is provided a method for treating depression by administering stable pharmaceutical compositions of bupropion hydrochloride of the present invention to a patient in need thereof.

Preferably, the stabilized oral pharmaceutical composition of bupropion hydrochloride is contained in specialized packaging materials, which protect the composition from moisture and oxygen. For example, suitable packaging materials include light protected high-density polyethylene bottles, light protected glass bottles and the like. Packaging will include a desiccant pack. The container may in the form of various types of blister packs to provide the desired protection, maintain product stability and integrity. It may be advisable to do packaging under nitrogen or argon to reduce the headspace oxygen. Preferably, the pharmaceutical compositions of bupropion hydrochloride of the present invention may be packed in HDPE container with molecular sieve sachets/canister and/or the container may be purged with nitrogen gas in order to provide oxygen free environment.

The following examples are provided to enable one of ordinary skill in the art to prepare dosage forms of the invention and should not be construed as limiting the scope of the invention.

### Example 1

TABLE 1
Bupropion hydrochloride sustained release tablets

| | | |
|---|---|---|
| S. No | Ingredients | Qty (mg/tab) |

(A) Core

| | | |
|---|---|---|
| 1. | Bupropion hydrochloride | 150.0 |
| 2. | Hydrogenated castor oil | 147.50 |
| 3. | Mannitol | 27.0 |
| 4. | Polyvinylpyrrolidone | 13.0 |
| 5. | Crospovidone | 6.5 |
| 6. | Magnesium stearate | 4.0 |
| 7. | Isopropyl alcohol* | QS |

| | |
|---|---|
| Total | 348.0 |

(B) Seal coating

| | | |
|---|---|---|
| 8. | Hydroxypropyl methylcellulose | 6.32 |
| 9. | Isopropyl alcohol* | QS |
| 10. | Dichloromethane* | QS |
| 11. | Polyethylene glycol | 0.63 |

| | |
|---|---|
| Total | 354.95 |

(C) Film coating

| | | |
|---|---|---|
| 12. | Opadry II | 14.2 |
| 13. | Purified water* | QS |

| | |
|---|---|
| Total | 369.15 |

*Evaporates while processing.

### Manufacturing Process:

Relevant steps were carried out under 25° C.±5° C. temperature and 25%±5% relative humidity.

### (A) Core Formation

a) Bupropion hydrochloride and hydrogenated castor oil were sifted through suitable screen.
b) Mannitol and crospovidone were sifted through suitable screen.
c) Magnesium stearate was sifted through suitable screen.
d) 11% solution of polyvinylpyrrlidone was prepared in isopropyl alcohol.
e) The powder mass of step (a) was mixed geometrically with powder mass of step (b).
f) The powder mass of step (e) was further mixed in suitable equipment.
g) The powder mass of step (f) was granulated with binder solution of step (d).
h) Granules of step (g) were dried in a suitable dryer and dried granules were milled through suitable mill.
i) Dried granules of step (h) were lubricated by mixing with magnesium stearate of step (c).
j) Lubricated granules of step (i) were compressed into tablet.

### (B) Seal Coating

k) Core tablets as obtained in step (j) were coated using above-mentioned seal coating composition and the coated tablets were cured at 40° C. for two hours.

### (C) Film Coating

l) Seal coated tablets as obtained in step (k) above were coated with the film-coating composition as mentioned above and the coated tablets were cured at 40° C. for two hours.

### (D) Packaging

m) Film coated tablets as obtained in step (l) above were packed in light protected HDPE bottles provided with molecular sieve/canister purged with nitrogen gas.

### Example 2

TABLE 2
Bupropion hydrochloride sustained release tablets

| | | |
|---|---|---|
| S. No | Ingredients | Qty (mg/tab) |

(A) Core

| | | |
|---|---|---|
| 1. | Bupropion hydrochloride | 150.0 |
| 2. | Hydrogenated castor oil | 178.00 |
| 3. | Mannitol | 25.00 |
| 4. | Polyvinylpyrrolidone | 15.00 |
| 5. | Crospovidone | 2.0 |
| 6. | Sodium Starch Glycolate | 3.0 |
| 7. | Magnesium stearate | 4.6 |
| 8. | Isopropyl alcohol* | QS |

| | |
|---|---|
| Total | 377.6 |

(B) Seal coating

| | | |
|---|---|---|
| 8. | Hydroxypropyl methylcellulose | 7.01 |
| 9. | Isopropyl alcohol* | QS |
| 10. | Dichloromethane* | QS |
| 11. | Polyethylene glycol | 0.701 |

| | |
|---|---|
| Total | 385.311 |

(C) Film coating

| | | |
|---|---|---|
| 12. | Opadry II | 15.48 |
| 13. | Purified water* | QS |

| | |
|---|---|
| Total | 400.791 |

*Evaporates while processing

### Manufacturing Process:

Relevant steps were carried out under 25° C.±5° C. temperature and 25%±5% relative humidity.

### (A) Core Formation

a) Bupropion hydrochloride and hydrogenated castor oil were sifted through suitable screen.
b) Mannitol, crospovidone and sodium starch glycolate were sifted through suitable screen.
c) Magnesium stearate was sifted through suitable screen.
d) 12% solution of polyvinylpyrrlidone was prepared in isopropyl alcohol.
e) The powder mass of step (b) was mixed geometrically with powder mass of step (a).
f) The powder mass of step (e) was further mixed in suitable equipment.
g) The powder mass of step (f) was granulated with binder solution of step (d).
h) Granules of step (g) were dried in a suitable dryer and dried granules were milled through suitable mill.
i) Dried granules of step (h) were lubricated by mixing with magnesium stearate of step (c).
j) Lubricated granules of step (i) were compressed into tablet.

### (B) Seal Coating

k) Core tablets as obtained in step (j) were coated using above-mentioned seal coating composition and the coated tablets were cured at 40° C. for two hours.

### (C) Film Coating

l) Seal coated tablets as obtained in step (k) above were coated with the film-coating composition as mentioned above and the coated tablets were cured at 40° C. for two hours.

### (D) Packaging

m) Film coated tablets as obtained in step (l) above were packed in light protected HDPE bottles provided with molecular sieve/canister purged with nitrogen gas.

### Example 3

TABLE 3
Bupropion hydrochloride sustained release tablets

| | | |
|---|---|---|
| S. No | Ingredients | Qty (mg/tab) |

(A) Core

| | | |
|---|---|---|
| 1. | Bupropion hydrochloride | 150.0 |
| 2. | Hydrogenated castor oil | 146.00 |
| 3. | Mannitol | 25.00 |
| 4. | Polyvinylpyrrolidone | 13.00 |
| 5. | Crospovidone | 7.0 |
| 6. | Magnesium stearate | 4.0 |
| 7. | Isopropyl alcohol* | QS |

| | |
|---|---|
| Total | 345.00 |

(B) Seal coating

| | | |
|---|---|---|
| 8. | Hydroxypropyl methylcellulose | 6.27 |
| 9. | Isopropyl alcohol* | QS |
| 10. | Dichloromethane* | QS |
| 11. | Polyethylene glycol | 0.627 |

| | |
|---|---|
| Total | 351.90 |

(C) Film coating

| | | |
|---|---|---|
| 12. | Opadry II | 13.8 |
| 13. | Purified water* | QS |

| | |
|---|---|
| Total | 365.70 |

*Evaporates while processing

### Manufacturing Process:

Relevant steps were carried out under 25° C.±5° C. temperature and 25%±5% relative humidity.

### (A) Core Formation

a) Bupropion hydrochloride and hydrogenated castor oil were sifted through suitable screen.
b) Mannitol and crospovidone were sifted through suitable screen.
c) Magnesium stearate was sifted through suitable screen.
d) 11% solution of polyvinylpyrrlidone was prepared in isopropyl alcohol.
e) The powder mass of step (b) was mixed geometrically with powder mass of step (a).
f) The powder mass of step (e) was further mixed in suitable equipment.
g) The powder mass of step (f) was granulated with binder solution of step (d).
h) Granules of step (g) were dried in a suitable dryer and dried granules were milled through suitable mill.
i) Dried granules of step (h) were lubricated by mixing with magnesium stearate of step (c).
j) Lubricated granules of step (i) were compressed into tablet.

### (B) Seal Coating

k) Core tablets as obtained in step (j) were coated using above-mentioned seal coating composition and the coated tablets were cured at 40° C. for two hours.

### (c) Film Coating

l) Seal coated tablets as obtained in step (k) above were coated with the film-coating composition as mentioned above and the coated tablets were cured at 40° C. for two hours.

### (D) Packaging

m) Film coated tablets as obtained in step (l) above were packed in light protected HDPE bottles provided with molecular sieve/canister and purged with nitrogen gas.

### Example 4

TABLE 4
Bupropion hydrochloride sustained release tablets

| | | |
|---|---|---|
| S. No | Ingredients | Qty (mg/tab) |

(A) Core

| | | |
|---|---|---|
| 1. | Bupropion hydrochloride | 150.0 |
| 2. | Hydrogenated castor oil | 180.0 |
| 3. | Mannitol | 25.00 |
| 4. | Polyvinylpyrrolidone | 15.20 |
| 5. | Crospovidone | 5.0 |
| 6. | Magnesium stearate | 4.8 |
| 7. | Isopropyl alcohol* | QS |

| | |
|---|---|
| Total | 380.0 |

(B) Seal coating

| | | |
|---|---|---|
| 8. | Hydroxypropyl methylcellulose | 6.81 |
| 9. | Isopropyl alcohol* | QS |
| 10. | Dichloromethane* | QS |
| 11. | Polyethylene glycol | 0.681 |

| | |
|---|---|
| Total | 387.49 |

(C) Film coating

| | | |
|---|---|---|
| 12. | Opadry II | 15.4 |
| 13. | Purified water* | QS |

| | |
|---|---|
| Total | 402.891 |

*Evaporates while processing

### Manufacturing Process:

Relevant steps were carried out under 25° C.±5° C. temperature and 25%±5% relative humidity conditions.

### (A) Core Formation

a) Bupropion hydrochloride and hydrogenated castor oil were sifted through suitable screen.
b) Mannitol and crospovidone were sifted through suitable screen.
c) Magnesium stearate was sifted through suitable screen.
d) 12% solution of polyvinylpyrrlidone was prepared in isopropyl alcohol.
e) The powder mass of step (b) was mixed geometrically with powder mass of step (a).
f) The powder mass of step (e) was further mixed in suitable equipment.
g) The powder mass of step (f) was granulated with binder solution of step (d).
h) Granules of step (g) were dried in a suitable dryer and dried granules were milled through suitable mill.
i) Dried granules of step (h) were lubricated by mixing with magnesium stearate of step (c).
j) Lubricated granules of step (i) were compressed into tablet.

### (B) Seal Coating

k) Core tablets as obtained in step (j) were coated using above-mentioned seal coating composition and the coated tablets were cured at 40° C. for two hours.

### (C) Film Coating

l) Seal coated tablets as obtained in step (k) above were coated with the film-coating composition as mentioned above and the coated tablets were cured at 40° C. for two hours.

### (D) Packaging

m) Film coated tablets as obtained in step (l) above were packed in light protected HDPE bottles provided with molecular sieve/canister purged with nitrogen gas.

### Example 5

To assess the release of drug substance (bupropion hydrochloride) from the drug product or dosage form, coated tablet of Example 1 was subjected to dissolution study. The dissolution profile from coated tablet of Example 1 was compared with the dissolution profile from the commercially available bupropion sustained release tablets (Wellbutrin®SR 150 mg) from Glaxosmithkline, USA. The results are presented in table 5 as a mean percentage release of the total bupropion hydrochloride contents from the coated tablets. Dissolution study parameters were as follows:

Instrument parameters: USP type I; 50 RPM
Dissolution parameters: 0.1 HCL, 900 ml, 37° C.±0.5° C.

TABLE 5
Dissolution profile

Mean percentage release of drug (bupropion)

| Time (hr) | Wellbutrin ®SR | Coated tablets of Example 1 |
|---|---|---|
| 0.5 | 19 | 26 |
| 1 | 29 | 39 |
| 2 | 44 | 57 |
| 4 | 65 | 83 |
| 6 | 79 | 102 |
| 8 | 88 | 102 |
| 10 | 95 | 102 |
| 12 | 97 | 102 |

From the above tabular data, it is clearly evident that there is no significant difference between dissolution profile of coated tablets of the invention (Ex. 1) and Wellbutrin®SR (reference product).

### Example 6

In order to assess the stability of drug substances (bupropion hydrochloride) in the drug product or dosage form, coated tablets of Example 1 to 4 and Wellbutrin®SR was subjected to accelerated stability testing at 40° C.±2° C./75% RH±5% RH and observations were made during and after three months in HDPE bottles having canister/molecular sieve/desiccant and nitrogen flushing for percentage of undegraded bupropion hydrochloride. Analysis was carried out by validated high performance liquid chromatography. The results are shown in Table 6 below:

TABLE 6
Accelerated stability data

| | | Coated | Coated | Coated | Coated |
|---|---|---|---|---|---|
| | Wellbutrin ® | tablets of | tablets of | tablets of | tablets of |
| Study | SR | Example 1 | Example 2 | Example 3 | Example 4 |
| period | % of undegraded Bupropion Hydrochloride present | | | | |
| 0 | 99.3 | 98.7 | NA | NA | 93.7 |
| 1 month | 96.3 | 97.8 | 97.3 | 95.3 | 100.0 |
| 3 month | 98.1 | 101.4 | 96.2 | 98.7 | 99.1 |

The results in the table above clearly depict that the content of undegraded bupropion hydrochloride in the compositions of the present invention in the form of sustained release tablets (Example 1, 2, 3 and 4) are comparable to the content of undegraded bupropion hydrochloride in the commercially available Wellbutrin®SR tablets.

### Example 7

In order to assess the stability of drug substances (bupropion hydrochloride) in the drug product or dosage form, compositions of Example 1 to 4 and Wellbutrin® SR were subjected to accelerated stability testing at 40° C.±2° C./75% RH±5% RH and observations were made during and after 3 months in HDPE bottles for the levels of m-chlorobenzoic acid. Analysis was carried out by high performance liquid chromatography. The results are shown in Table 7 below:

TABLE 7
Accelerated stability data

| | | Coated | Coated | Coated | Coated |
|---|---|---|---|---|---|
| | Wellbutrin ® | tablets of | tablets of | tablets of | tablets of |
| Study | SR | Example 1 | Example 2 | Example 3 | Example 4 |
| period | m-chlorobenzoic acid (% w/w) | | | | |
| 0 | 0.05 | 0.04 | NA | NA | 0.09 |
| 1 month | 0.05 | 0.08 | 0.09 | 0.05 | 0.07 |
| 3 month | 0.04 | 0.13 | 0.15 | 0.13 | 0.16 |

The results of analysis clearly depict that even in the absence of acid stabilizer or any other kind of stabilizer, level of m-chlorobenzoic acid in the composition of e.g. 1 to 4 is below 0.3% and is comparable with Wellbutrin SR which employs substantial amount of acid stabilizer.

### Example 8

This example demonstrates the ability of formulation of example 1 (labeled amount 150 mg bupropion) to provide bioavailability of bupropion, which is comparable to bioavailability of Wellbutrin®SR (labeled amount of 150 mg of bupropion) as determined by the area under the curve (AUC) and Cₘₐₓ.

This example describes an in-vivo study, which measured plasma concentrations of bupropion achieved after oral administration of reference Wellbutrin® SR tablet and test bupropion hydrochloride sustained release tablets of example 1. The in-vivo study was carried out on 12+2 standby healthy adult subjects under fasting conditions. Plasma bupropion was determined over a 120 hour period, after single oral administration of the respective formulations. Each subject was administered each of the two formulations in cross over design separated by a washout period of 14 days between administrations of the two formulations. Plasma levels were measured at predetermined times utilizing a validated assay method employing LC-MS/MS instrumentation. Plasma pharmacokinetic parameters were evaluated using commercially available software WinNonlin version 5.0.1 or higher. The statistical analysis was performed using SAS® package (SAS® Institute Inc., USA version 9.1 or higher). Values derived at each time point and to determine the area under the curve (AUC) and maximum concentration (Cmax) afforded by Wellbutrin®SR and the test formulation. The test formulation provided bioavailability of bupropion, which is substantially equivalent to that of Wellbutrin® SR, as determined by the software program. Pharmacokinetic parameters for both reference and test formulation have been shown in table:

TABLE 8
Pharmacokinetic Data

| | Reference product | Test formulation | | 90% confidence |
|---|---|---|---|---|
| Parameter | (Wellbutrin ®SR)-R | (Example 1)-T | T/R ratio | interval (T Vs R) |
| AUC₀₋ₜ(ngh/ml) | 894.88 | 861.852 | 99.22 | 90.18-109.17 |
| AUC_{0-α}(ngh/ml) | 951.251 | 918.649 | 99.44 | 90.38-109.40 |
| Cₘₐₓng/ml | 111.178 | 116.019 | 106.52 | 94.96-119.48 |
| Tₘₐₓ | 3.958 | 3.792 | — | — |

In view or the above, it is clearly evident that formulation of the invention importantly provide bioavailability of bupropion, which is comparable, or bioequivalent to Wellbutrin®SR.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the scope of the present invention. Moreover, it is contemplated that any single feature or any combination of optional features of the inventive variations described herein may be specifically excluded from the claimed invention and be so described as a negative invention. Accordingly, it is not intended that the invention be limited, except as by the appended claims.

1. An oral stable sustained release pharmaceutical composition comprising therapeutically effective amount of uncoated fine bupropion hydrochloride and pharmaceutically acceptable adjuvants, wherein the composition is free of an acidic stabilizer and contains less than about 0.3% by weight of m-chlorobenzoic acid when stored at 40° C.±2° C. and 75%±5% RH for three months in specialized packs.

2. An oral stable sustained release pharmaceutical composition comprising therapeutically effective amount of uncoated fine bupropion hydrochloride and pharmaceutically acceptable adjuvants, wherein the composition is free of an acidic stabilizer and contains less than about 0.3% by weight of m-chlorobenzoic acid when stored at 40° C.±2° C. and 75%±5% RH for three months in specialized packs and wherein said composition is manufactured under controlled processing conditions.

3. The pharmaceutical composition according to claim 1, wherein the fine bupropion hydrochloride having particles size in a range of about 1 to 100 μm.

4. The pharmaceutical composition according to claim 1, wherein the composition further contains 25% to 55% of lipophilic release retardant.

5. The pharmaceutical composition according to claim 4, wherein said lipophilic release retardant(s) are selected from one or more hydrogenated oils comprising from hydrogenated vegetable oil, cottonseed oil, castor oil, canola oil, palm oil, palm kernel oil and soybean oil, cetostearyl alcohol, cetyl alcohol, glyceryl behenate derivatives, glyceryl mono oleate, glyceryl mono stearates, glyceryl palmito stearates, lecithin, mono-di- and triglycerides with polyethylene glycol (PEG) esters of fatty acid, medium chain triglycerides, carnauba wax, microcrystalline wax, beeswax or any combination thereof.

6. The pharmaceutical composition according to claim 5, wherein the lipophilic release retardant is preferably hydrogenated castor oil.

7. The pharmaceutical composition according to claim 1, wherein the pharmaceutically acceptable adjuvants are selected from one or more weight-adjusting agents or channeling agents, swelling enhancers, binders, lubricants, glidants, or any combination thereof.

8. The pharmaceutical composition according to claim 1, wherein said specialized packs includes HDPE bottles with molecular sieve sachets or blister packs, purged with nitrogen gas.

9. The pharmaceutical composition of claim 1, wherein the composition contains more than 85% of undegraded bupropion hydrochloride after storage for three months at 40° C.±2° C. and 75%±5% RH.

10. The pharmaceutical composition according to claim 1, wherein the composition is formulated in various oral delivery devices, preferably tablet, capsule, granules, beads, or sachet.

11. The pharmaceutical composition according to claim 1, wherein the composition is prepared under controlled processing conditions comprising the steps of: (a) preparing a core, (b) optionally, forming a seal coating layer on the core and (c) optionally, forming a film coating on the seal coated core, and (d) packing final product in specialized packs.

12. The pharmaceutical composition according to claim 11, wherein said controlled processing conditions involve relative humidity maintained at about 25%±5% and temperature maintained at about 25° C./±5° C.

13. The pharmaceutical composition according to claim 12, wherein the process comprising the steps of:
(a) preparing the core comprising:
(i) sifting bupropion hydrochloride and lipophilic release retardant material(s),
(ii) sifting channeling agent(s) and swelling enhancer(s) and optionally other pharmaceutical adjuvant (s),
(iii) preparing a binder solution in either aqueous or non-aqueous solution,
(iv) mixing the blend of step (i) geometrically with blend of step (ii),
(v) granulating the resultant blend employing aqueous or non-aqueous solvent,
(vi) drying the resultant granulates,
(vii) lubricating the dried granulates with lubricant,
(viii) compressing the lubricated granulates into a compressed core;
(b) optionally forming the seal-coating layer on the core;
(c) optionally forming the non-functional film coating on the seal coated core; and
(d) packing the resultant product in specialized packs.

14. The pharmaceutical composition according to claim 2, wherein the composition is prepared under controlled processing conditions comprising the steps of: (a) preparing a core, (b) optionally, forming a seal coating layer on the core and (c) optionally, forming a film coating on the seal coated core, and (d) packing final product in specialized packs.

15. The pharmaceutical composition according to claim 14, wherein said controlled processing conditions involve relative humidity maintained at about 25%±5% and temperature maintained at about 25° C.±5° C.

16. The pharmaceutical composition according to claim 12, wherein the process comprising the steps of:
(a) preparing the core comprising:
(i) sifting bupropion hydrochloride and lipophilic release retardant material(s),
(ii) sifting channeling agent(s) and swelling enhancer(s) and optionally other pharmaceutical adjuvant (s),
(iii) preparing a binder solution in either aqueous or non-aqueous solution,
(iv) mixing the blend of step (i) geometrically with blend of step (ii),
(v) granulating the resultant blend employing aqueous or non-aqueous solvent,
(vi) drying the resultant granulates,
(vii) lubricating the dried granulates with lubricant,
(viii) compressing the lubricated granulates into a compressed core;
(b) optionally forming the seal-coating layer on the core;
(c) optionally forming the non-functional film coating on the seal coated core; and
(d) packing the resultant product in specialized packs.
